(19) United States
(12) Patent Application Publication
(10) Pub. No.: US 2024/0127958 A1
(43) Pub. Date: Apr. 18, 2024

(54) METHOD FOR DISEASE RISK ASSESSMENT

(71) Applicant: PI BIOELECTRONICS COMPANY LIMITED, Taichung City (TW)

(72) Inventors: Min-Hui ChiouChang, Taichung City (TW); Yung-Jiun Lin, Taichung City (TW); Wei-Ting Hsieh, Taichung City (TW)

(21) Appl. No.: 17/965,818

(22) Filed: Oct. 14, 2022

(51) Int. Cl. G16H 50/30; G16H 10/60; G16H 40/20

(52) U.S. Cl. CPC G16H 50/30; G16H 10/60; G16H 40/20

(57) ABSTRACT

A method for disease risk assessment includes a data acquiring step, a preprocessing step, and a determining step. In the data acquiring step, a medical record of a subject, a static physiological information of the subject measured, and a dynamic physiological information corresponding to different actions of the subject are obtained. In the preprocessing step, a terminal device is applied for integrating the aforementioned data to generate a current data. In the determining step, the current data is inputted into a prediction model for calculation, so as to generate a disease risk assessment result corresponding to the subject. The assessment result includes a disease category, an onset probability corresponding to the disease category, and an estimated time of the onset of the disease. Thus, the present invention accurately assesses the disease probability of the subject in the future for health improvement and disease prevention and postponement.

### BACKGROUND OF THE INVENTION

### 1. Field of the Invention

The present invention relates to risk assessment techniques, and more particularly, to a method for disease risk assessment.

### 2. Description of the Related Art

Currently existing disease risk assessment system aims at analyzing the static physiological data, living habits, and family health history of the subjects to obtain a disease risk assessment based on the health examination reports of the subjects or the questionnaires filled out by the subjects.

However, the examination data reports generally obtained from the health examination provided by the health examination center or other medical units only reflect the current health status of the subject, such that the examination data reports facilitate a relatively limited pathological judgements in clinical practice, such as for reference of further examination on the bases of liver function or renal function abnormality, etc. Also, the normal range of relative data are often universal without differences. In other words, the normal range of reference data values is applied for all the subjects, regardless of genders and ages. For example, the normal range of the blood pressure values is identically set as 120/80 mmHg. The conventional health risk assessment method is unable to reflect a customized assessment result with respect to individually different conditions.

Also, early symptoms of many diseases will be manifested in the changes of body motions and reactions. The abnormality of the nervous system of the subjects is unable to be detected by simply referring to the health examination data reports and questionnaires filled out by the subjects. Therefore, it is impossible to accordingly assess the risk of relative diseases, improve the health status, and prevent diseases in advance by the conventional health examination methods.

### SUMMARY OF THE INVENTION

The present invention aims at assessing the possibility of specific diseases which might be suffered by the subject in the future through a periodically and long-term health trend assessment, thereby reminding the subject to improve the health condition in advance for preventing or postponing the possible diseases. More particularly, the present invention applies a periodical assessment of dynamic features of specific motions and movements carried out by the subject for assessing the possibility of diseases featuring action disability which the subject might be suffered, so as to remind the subject to improve the health condition in advance for preventing or postponing the possible diseases.

For achieving the aforementioned objectives, the present invention provides a method for disease risk assessment, comprising a data acquiring step, a preprocessing step, and a determining step. In the data acquiring step, a data acquisition module is applied for obtaining a medical record, a static physiological information of the subject measured, and a dynamic physiological information corresponding to different actions of the subject. The static physiological information is obtained through a static physiological detection instrument measuring the physiological characteristics of the subject when the subject rests or stops moving. The dynamic physiological information is obtained by giving a plurality of action commands to the subject and measuring the physiological characteristic of the subject corresponding to each action command carried out by the subject through a dynamic physiological detection instrument. In the preprocessing step, a terminal device is applied for integrating the aforementioned data to generate a current data. In the determining step, the current data is inputted into a prediction model for calculation, so as to generate a disease risk assessment result corresponding to the subject. The assessment result includes a disease category, an onset probability corresponding to the disease category, and an estimated time of the onset of the disease.

Therefore, the prediction model is trained and established by inputting the medical record, static physiological information, and dynamic physiological information of a patient who has been suffering specific diseases, especially including the medical record, static physiological information, and dynamic physiological information of the patient both before and during the diagnosis of the diseases. When a subject, who has not previously been diagnosed with the disease, is diagnosed with the disease, the past and current data of the corresponding subject will become a new training case for updating the parameters of the prediction model.

With such configuration, the method for disease risk assessment of the present invention captures the medical record and the measured static and dynamic physiological information of the subject, and integrates such data into a current data, which is inputted into the prediction model, thereby generally analyzing the past and current health conditions of the subject and the activity and body control capability of the subject, so as to obtain the assessment result of the disease risk of the subject in the future. Thus, the present invention obtains the variation trend of future health conditions of the subject, allowing the subject to improve the health condition in advance for preventing or postponing the possible diseases.

### BRIEF DESCRIPTION OF THE DRAWINGS

FIG. 1 is a structural block view of the assessment system in accordance with an embodiment of the present invention.

FIG. 2 is a flow chart of the method for disease risk assessment in accordance with an embodiment of the present invention.

FIG. 3 is a flow chart of the system in accordance with an embodiment of the present invention.

### DETAILED DESCRIPTION OF THE INVENTION

The aforementioned and further advantages and features of the present invention will be understood by reference to the description of the preferred embodiment in conjunction with the accompanying drawings where the components are illustrated based on a proportion for explanation but not subject to the actual component proportion.

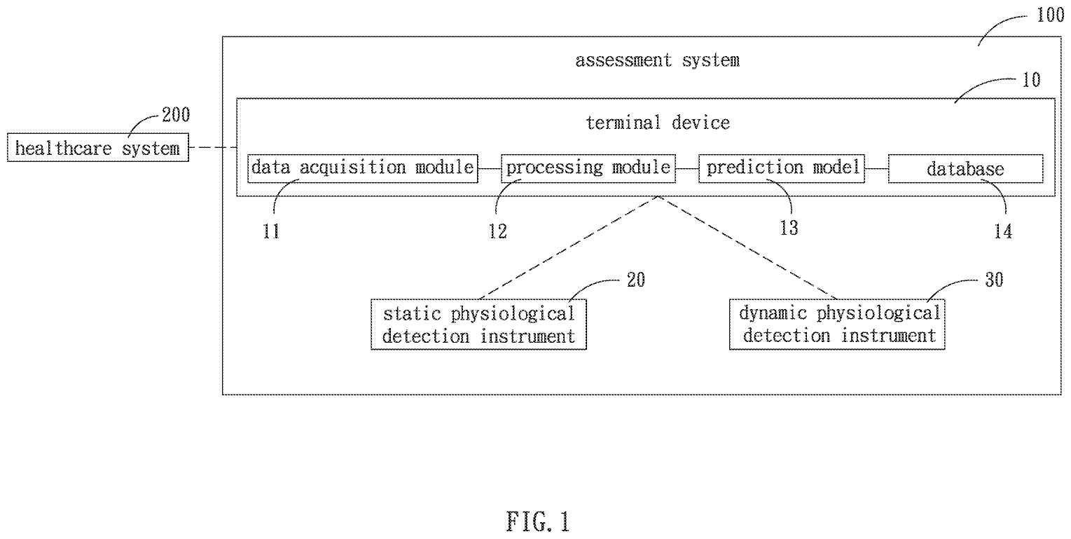

Referring to FIG. 1 to FIG. 3, the present invention provides a method for disease risk assessment, which is carried out through an assessment system 100.

The assessment system 100 comprises a terminal device 10, a static physiological detection instrument 20, and a dynamic physiological detection instrument 30. As shown by FIG. 1, the terminal device 10 is coupled with the static physiological detection instrument 20, the dynamic physiological detection instrument 30, and a healthcare system 200.

The terminal device 10 is configured to obtain a medical record of a subject. The static physiological detection instrument 20 is configured to obtain a static physiological information by measuring the physiological characteristics of the subject when the subject stops moving or acting. The dynamic physiological detection instrument 30 is configured to obtain a dynamic physiological information by measuring the physiological information of the subject when the subject executes different actions.

In the embodiment, the terminal device 10 is allowed to be, for example but not limited to, various types of cloud computing devices, personal computers, laptops, smart mobile devices, or tablet computers. The static physiological detection instrument 20 is allowed to be, for example but not limited to, a blood glucose meter, plasma drug concentration sensor, clinical thermometer, blood pressure meter, body weight scale, body height meter, hand dynamometer, dioptometer, audiometer, measuring tape (for measurement of chest circumference, abdominal circumference, leg circumference), body fat meter, electroencephalogram machine, electrocardiogram machine, and oximeter, and a combination of the instruments above. The dynamic physiological detection instrument 30 is allowed to be, for example but not limited to, a clinical thermometer, blood pressure meter, oximeter, electrocardiogram machine, electroencephalogram machine, electromyogram machine, plantar pressure sensor, and inertial measurement system, motion capture system or camera device, and a combination of the instruments above.

In the embodiment, the medical record includes the historical Anatomical Therapeutic Chemical Classification (ATC) codes and prescriptions of the subject. Also, the medical record further includes the date of diagnosis, the name of the diagnosed diseases, and the International Classification of Diseases Code (ICD-10) for the name of the diagnosed diseases of the subject.

In the embodiment, the static physiological information is selected from the blood test value, body temperature, weight, height, body fat rate, hand grip strength, chest circumference, abdominal circumference, leg circumference, blood pressure, blood oxygen, vision, hearing, blood sugar, static electrocardiogram, or static electroencephalogram of the subject or a combination thereof.

In the embodiment, the dynamic physiological information includes a plurality of time domain signals representing different physiological characteristics. The time domain signals are selected from continuous body temperature detection, continuous blood pressure detection, continuous blood oxygen detection, electrocardiography signal, electroencephalography signal, electromyography signal, plantar pressure signal, body torso motion, inertia signal, motion capture signal, or image signal of the subject, or a combination thereof.

The terminal device 10 comprises a data acquisition module 11, a processing module 12, and prediction model 13, and a database 14 that are coupled with each other. The data acquisition module 11 is connected with the healthcare system 200 through the internet for obtaining the medical record of the subject. The data acquisition module 11 is also communicatively connected with the static physiological detection instrument 20 and the dynamic physiological detection instrument 30 for obtaining the static physiological information and the dynamic physiological information. The processing module 12 is configured to integrate the medical record, the static physiological information, and the dynamic physiological information of the subject to generate a current data and input the current data and the past data of the identical subject previously stored in the database 14 into the prediction model 13, so as to carry out the analysis and obtain a disease risk assessment result. Therein, the disease risk assessment result includes a disease category, an onset probability corresponding to the disease category, and an estimated time of the onset of the disease. The current data is stored in the database 14, such that when the subject carries out the prediction next time, the current data serves as the past data for the analysis of the prediction model 13 or as the update training data of the prediction model 13 in the future.

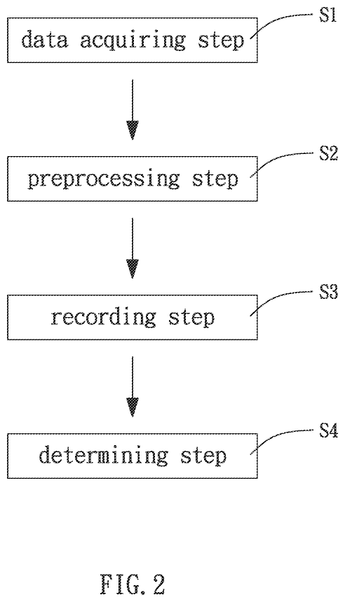

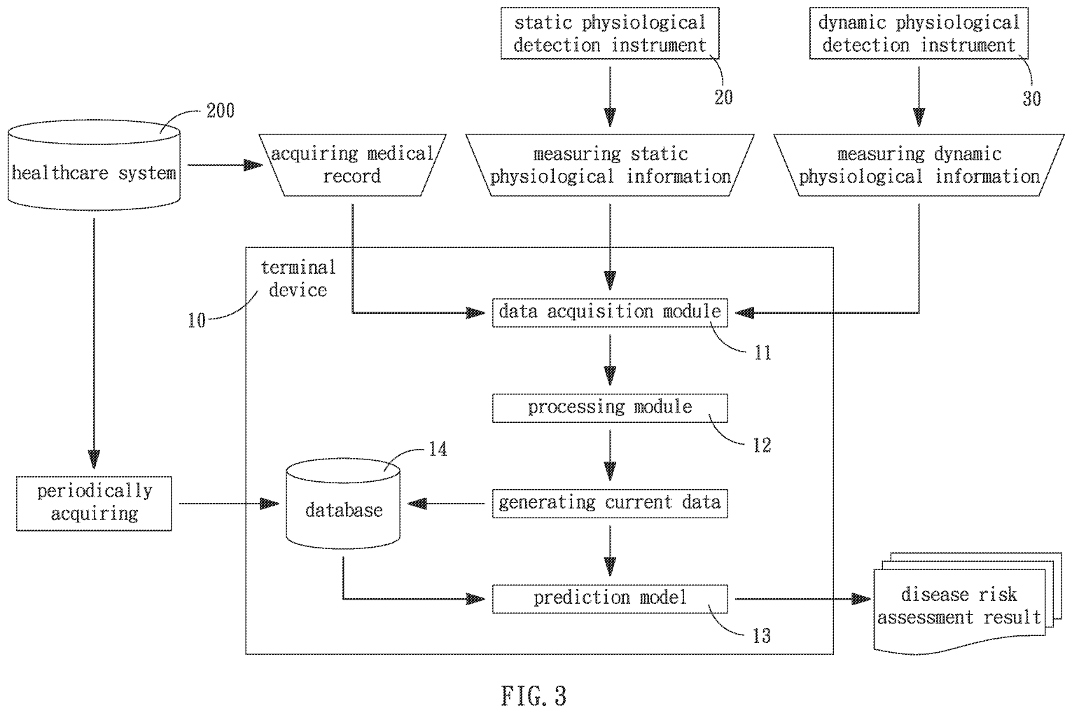

The content above is an embodiment of the assessment system 100 of the present invention. Referring to FIG. 2 to FIG. 3, the method for disease risk assessment of the present invention is illustrated as follows. FIG. 2 is a flow chart of the method for disease risk assessment in accordance with an embodiment of the present invention. FIG. 3 is a flow chart of system of disease risk assessment in accordance with an embodiment of the present invention. The method for disease risk assessment comprises a data acquiring step S1, a preprocessing step S2, a recording step S3, and a determining step S4.

In the data acquiring step S1, the data acquisition module 11 is connected with the healthcare system 200 to obtain the last medical record of the subject. The static physiological detection instrument 20 is applied for measuring the static physiological characteristics of the subject when the subject rests or stops moving or acting (such as standing, sitting, or lying still), so as to obtain the static physiological information. Also, the present invention gives a plurality of action commands to the subject and measures the dynamic physiological characteristics of the subject corresponding to each action command carried out by the subject through a dynamic physiological detection instrument 30, so as to obtain the dynamic physiological information.

In the embodiment, the action command given to the subject is selected from standing on one leg with eyes open, standing on two legs with eyes open, standing on two legs in a tandem stance with eyes open, standing on tiptoes with eyes open, standing on one leg with eyes closed, standing on two legs with eyes closed, standing on two legs in a tandem stance with eyes closed, standing on tiptoes with eyes closed, standing up, sitting down, squatting, walking in a straight line, turning, and climbing steps, and a combination thereof.

In the preprocessing step S2, the processing module 12 of the terminal device 10 integrates the medical record, the static physiological information, and the dynamic physiological information of the subject to generate a current data. The current data is generated by the processing module 12 carrying out a synchronization process and characteristic analysis with the medical record, the static physiological information, and the dynamic physiological information.

Notably, during the execution of a single action command, such as walking in a straight line, the duration from the subject hearing the command of walking forward to taking the first step is regarded as one event. In the gait cycle after the subject starts walking, the left foot and the right foot leaving the ground and swinging are regarded as individual events. If the time of each event are not accurately marked, the time duration taken by an elder subject and a patient with early Parkinson's disease from hearing the command to completing the first step may be the same. However, the patient suffering the Parkinson's disease has a freezing of gait symptom, so that the time durations taken by the two subjects to take the first step are different. Therefore, in addition to marking the starting stage and the left and right foot swing stages to calculate the difference of duration, it is necessary to further distinguish the “starting,” the “left foot swing,” and the “right foot swing” as different events, and mark the time of those events on the electroencephalography signal, electromyography signal, plantar pressure signal, and the time signals of body motion image, so as to carry out an accurate correlation and variance analysis with each dynamic physiological information according to different events during the analysis process.

For further illustration, the event synchronization of dynamic physiological information is in the stage of dynamic physiological information measurement. When a specific event occurs, the specific event is immediately numbered or encoded as an event flag, and the event flag is then marked on the time domain signal data measured by each dynamic physiological information measuring equipment. The time point at which the event flag is generated is the moment when a command, action, or special event starts or ends, so that the time points at which the event is generated are consistent when each time domain signal undergoes the characteristic analysis process based on the event. The marking is allowed to be made by manually pressing the event marker, by a predesigned guidance device synchronously triggering the event marker during the guiding process, or by one dynamic physiological detection instrument 30 triggering the event marker, whereby the event marker emits a mark signal of an event flag, such that other dynamic physiological detection instruments 30 receive the mark signal and mark it in the respectively recorded dynamic physiological signals. Also, the processing module 12 is allowed to analyze the signal of a certain dynamic physiological detection instrument 30 first as an event flag mark.

Further, the characteristic analysis of the dynamic physiological information divides the time domain signal of each physiological information into a plurality of synchronization blocks according to the event flags of each event according to the sequence that the subject executing action commands. The block is able to be defined with two event flags, or a signal event flag plus certain seconds forward or backward, or a signal event flag as a middle point plus certain seconds forward and backward, and then generates a plurality of characteristic indexes based on the correlation, time difference, cycle variation, coordination, and trend of the shifting of the center of gravity between each synchronization block. Therein, the characteristic index is related to the muscle strength, the shifting ability of the center of gravity, the balance ability, the nerve sensation sensitivity, the coordination of nerves, and the speed of reaction of the subject when the subject voluntarily moves in each event stage. In the embodiment, the plurality of synchronization blocks respectively include a command receiving block, an action consciousness block, an initialization block, an acceleration block, a maintenance block, a deceleration block, a stopping block, and a consciousness ending block.

Notably, a voluntary action comes from the consciousness of action in the consciousness area of the brain, and the action consciousness drives the operation of the motor cortex of the brain of the subject, and then triggers the action area controlling the muscle to control the corresponding muscle group to produce the body movements and myoelectric reactions. Also, the sensation feedback area receives environmental information sensed by the sensory nervous system. Accordingly, the operation effect of the overall muscle group is largely reflected in the distribution of pressure and the variation of the center of gravity of the sole.

Therefore, the processing module 12 is able to combine the electroencephalography signal and electromyography signal of the dynamic physiological information to obtain the correlation and time difference between the muscles and the neuromotor activity of the brain cortex, thereby forming the characteristic index corresponding to the voluntary action of each event stage.

The processing module 12 is also able to obtain different gait cycle signals and compare them with the electromyography signal, so as to analyze the force outputting and timing conditions of the muscle groups and the trend of the plantar pressure variation which generates the shifting of the center of gravity, thereby generating the characteristic index related to the center of gravity.

Taking the Timed Up and Go (TUG) test as the example, the synchronization process and the characteristic analysis of the gait of the subject are illustrated. Assuming that the action commands sequence given to the subject starts from sitting on a chair to “getting up”, “walking in a straight line” to a cone at 3 meters away, “turning”, “walking in a straight line”, and returning to the original position and then “sitting down”. By use of the dynamic physiological detection instrument 30 such as a camera system, electromyogram-electroencephalogram coherence equipment, or plantar pressure detector, the dynamic physiological information of the subject carrying out the TUG test is obtained. Based on the camera signal, the processing module 12 analyzes the time point of each action of “getting up”, “walking in a straight line”, “turning”, and “sitting down” as the event flags, and then marks them in the signals of other dynamic physiological information. Therefore, the synchronization process of the time domain signal of each physiological characteristic of the overall dynamic physiological information is carried out, and each time domain signal of the actions of “getting up”, “walking in a straight line”, “turning”, and “sitting down” in each stage has a consistent time point for the signal analysis.

In the embodiment, the processing module 12 further analyzes the time domain signal of the action of “walking in a straight line” and divides it into the command receiving block, action consciousness block, initialization block, acceleration block, maintenance block, deceleration block, stopping block, and consciousness ending block.

Therein, the processing module 12 reestablishes image signals of the initialization block, acceleration block, maintenance block, deceleration block, and stopping block into a human skeleton, records the spatial movement of the subject from the visual perspective, further divides the movement area into a stance phase and swing phase, and combines the electromyography signal to obtain the force outputting and timing conditions of the muscle groups under different phases, thereby generating the characteristic index related to the limbs and trunk coordination.

In the embodiment, the processing module 12 further combines the electromyography signal and electroencephalography signal of the action consciousness block, initialization block, acceleration block, maintenance block, deceleration block, stopping block, and consciousness ending block, so as to obtain the correlation between the actions and the neuromotor activity of the brain cortex, thereby generating the characteristic index related to the nerve sensation sensitivity, the coordination of nerves, and the speed of reaction.

In the embodiment, the processing module 12 further obtains the trend of the shifting of the center of gravity of the subject according to the plantar pressure signal of the initialization block, acceleration block, maintenance block, deceleration block, and stopping block, thereby generating the characteristic index of the shifting of the center of gravity and the balance ability.

For further illustrations, the aging degrees or the health conditions of the subject can be analyzed based on the controlling ability of voluntary actions, the delaying time between the command giving and the generation of action, and the coordination degree of different actions. Also, from the activity ability of the subject, the severity of the movement disorder of the subject is able to be analyzed for facilitating the determination of the risk of subsequent diseases. Therefore, through the characteristic index generated from the characteristic analysis of the dynamic physiological information, the controlling ability of the voluntary actions and the activity of the subject can be further understood, so as to determine the risk of diseases of the subject.

In the preprocessing step S2, the processing module 12 further obtains the historical Anatomical Therapeutic Chemical Classification (ATC) codes and prescriptions of the subject and the name of diagnosed diseases and the international classification of disease codes from the medical record, so as to determine if the medicine taken by the subject affects the static physiological information and dynamic physiological information measured from the subject. On the other hand, by obtaining the ATC codes of the medicines from the medical treatment record, the to-be-treated issues of the subject are confirmed, which can be taken as the reference for the subsequent disease risk assessment.

In the recording step S3, the processing module 12 records the current data of the subject generated from the preprocessing step S2 each time, and stores them into the database 14 based on the date of the integration as the historical record of the subject.

In the determining step S4, the processing module 12 inputs the current data and the historical record of the subject into the prediction model 13 for calculation, so as to apply the historical record variation and the complete historical record data of the subject to assist the prediction model 13 in generating the disease risk assessment result, thereby preventing the usage of the data at a signal time point, which might lead to erroneous judgements.

For example, the subject is 58 years old currently and has used the assessment system 100 to carry out the disease risk assessment once per three months from the age of 52. Assuming that the subject had continued to take medicine due to a cold caught in the first two weeks of the period of the last examination, which results an abnormal liver function in the latest current data of the subject, because the historical records of the subject in the past has never shown an abnormal liver function, the prediction model 13 is able to exclude the assessment result related to the diseases caused by abnormal liver function, thereby avoiding misjudgment.

Also, when the current data is inputted into the prediction model 13, according to a characteristic weight corresponding to different diseases previously stored in the database 14, the prediction model 13 carries out a weight distribution of the current data and calculates the possible diseases and the probability of onset of the diseases. Therein, the characteristic weight comprises the weight parameters of a plurality of characteristic indexes of the medical record, static physiological information, and dynamic physiological information of the current data.

In the embodiment, the distribution principle of the characteristic weight is to distribute the characteristic parameters according to different diseases, and then increase or decrease the medication related weight parameters based on the medication data and prescriptions in the medical record of the subject, so as to lower the interference caused by the medication upon the subject, thereby carrying out a more accurate assessment of specific diseases.

For example, through the current level of technology, it is known that the factors highly correlated to stroke are “hypertension, obesity, cholesterol, and diastolic blood pressure”. Therefore, for assessment of stroke, the weight parameters of those factors can be appropriately increased, so as to conform with the actual situation. In fact, it is also possible that, through data collection, an AI is able to understand such trend and accordingly carry out an appropriate adjustment of the parameter model.

In the determining step S4, the prediction model 13 further compares the current data with the default case in the prediction model 13 for estimating the time of disease onset, so as to generate the disease risk assessment result. By comparing the characteristic index of the current data and the default case, the difference between the subject and the large group is obtained through the statistical results of the big data of cases, thereby assessing the estimated time of onset of the disease from the characteristic level established based on the cases. In the embodiment, the cases are related to the specific diseases and the execution of specific actions and are established based on further statistics and classification of race, country, gender, and age. For example, the gait characteristic of a walking subject is compared with that of a default stroke case. Assuming that the subject is a 66-year-old Asian man, when the prediction model 13 receives the current data of the subject, the prediction model 13 compares the characteristic index of the current data related to walking with the gait cases of 65- to 70-year-old Asian males for calculating the possible risk and time of the subject suffering stroke.

In the embodiment, the prediction model 13 is established by collecting dynamic physiological information, static physiological information, and medical record of a plurality of subjects whose medical record has clear disease classification codes during each assessment, so as to be established by use of a machine learning algorithm. Particularly, the prediction model 13 is applied for finding out the variation trend of various medical records, static physiological information, and dynamic physiological information before the time point when each disease is clearly diagnosed. When the variation of relative data of a new subject who has not yet suffered from the disease conforms to the trend and time course of the data of a known disease, the probability of the chance and estimated time of onset of the disease is allowed to be evaluated.

Regarding the update of the prediction model 13, by continuously adding a plurality of subjects who have not yet suffered from the disease, the medical record obtained by each subject connected to the healthcare system 200 through the data acquisition module 11 will detect if the disease classification code items have been updated. Regarding the subject with new or updated disease classification numbers, the medical record, current data, and detection history record in the past obtained from the database 14 will be used in the verification, retraining, and updating of the prediction model 13, so as to generate a continuously evolving prediction model 13. In particular, after the subject develops a major disease, the assessment system 100 may no longer be used for assessing this subject. Therefore, the database 14 will also periodically capture the medical records of all the subjects to determine if any subject suffers from a serious disease. The data of these types of subjects with major diseases are also one of the valuable data for the verification, retraining, and parameter updating of the prediction model 13.

For further illustration, the training of the prediction model 13 is combined with case-based reasoning technique. By continuously accumulating the subject cases who suffer from diseases, the variation trend of the physiological variation and the voluntary action ability of the subject having a clear disease mark is applied for adding the training data of the prediction model 13, allowing the prediction model 13 to continuously evolve and increasing the accuracy of the prediction model 13.

For example, a 66-year-old Asian female subject is assessed for Parkinson's disease through the evaluation system 100. Parkinson's disease is a chronic neurodegenerative disease which affects the central nervous system. The precursors of the onset of Parkinson's disease are the decline of motion function and gait abnormality during walking.

In the embodiment, the subject is assessed and evaluated through the assessment system 100 once per month since the age of 63. Therefore, in the data acquiring step S1, the latest medical record, the current static and dynamic physiological information are obtained. In the preprocessing step S2, the processing module 12 obtains the historical record of the subject in the past from the database 14 and integrates the static and dynamic physiological information of the subject to generate the current data. In the recording step S3, the processing module 12 records the current data of the subject into the database 14. In the determining step S4, the processing module 12 inputs the current data of the subject into the prediction model 13 for calculation. According to the historical record, the subject consumes the medication leading to rising the blood pressure every month from age 63 to age 66, and the subject has chronic prescription. Also, during the year from age 65 to age 66, the characteristic index related to gait presents two-centimeter reduction every three months, and the characteristic index related to shifting ability of the center of gravity presents a reducing trend, and the blood pressure during execution of different actions presents a gradually rising trend. With the calculation of the prediction model 13, it is known that the chance of the subject being diagnosed with Parkinson's disease in nine months is 80%. Based on the disease risk assessment result, the subject is allowed to receive medication treatment and carry out rehabilitation exercises in advance, so as to improve the activity function of the body and increase the living quality, thereby delaying the onset of Parkinson's disease.

To sum up, the method for disease risk assessment of the present invention captures the medical record of the subject and measures the static and dynamic physiological information to integrate them into the current data, so as to use the processing module 12 and the prediction model 13 to generally analyze the current and historical health conditions, the activity capability, and the body controlling capability of the subject, thereby obtaining the disease risk assessment result and understanding the variation trend of the health conditions of the subject in the future. Therefore, the subject is allowed to take early health control to improve the physical condition, reduce the risk of disease, and prevent or delay the occurrence of diseases.

Also, by continuously collecting a large amount of data from different subjects, especially the subject having a new disease diagnosed, the present invention keeps training and validating the prediction model 13, allowing the prediction model 13 to continuously evolve, and improving the disease determination capability thereof, allowing other subjects which have not yet been diagnosed with diseases to accurately assess the probability and the possible time of the onset of diseases.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A method for disease risk assessment, comprising:
a data acquiring step: using a data acquisition module to obtain a medical record, a static physiological information, and a dynamic physiological information corresponding to different actions of a measured subject, the static physiological information being obtained through a static physiological detection instrument measuring physiological characteristics of the subject when the subject is resting or not moving, the dynamic physiological information being obtained by giving a plurality of action commands to the subject and measuring physiological characteristics of the subject corresponding to each action command carried out by the subject through a dynamic physiological detection instrument;
a preprocessing step: using a terminal device to integrate the medical record, the static physiological information, and the dynamic physiological information to generate a current data; and
a determining step: inputting the current data into a prediction model for calculation, so as to generate a disease risk assessment result corresponding to the subject, the disease risk assessment result including a disease category, an onset probability corresponding to the disease category, and an estimated time of the onset of the disease.

2. The method for disease risk assessment of claim 1, wherein in the preprocessing step, the terminal device carries out a characteristic analysis of the medical record, the static physiological information, and the dynamic physiological information to generate the current data.

3. The method for disease risk assessment of claim 2, wherein in the preprocessing step, the terminal device carries out a synchronization process of the dynamic physiological information; the dynamic physiological information comprises a plurality of time domain signals representing different physiological characteristics when an event occurs, and the terminal device immediately and synchronously records the event in a numbering or encoding manner as an event flag in each time domain signal.

4. The method for disease risk assessment of claim 3, wherein the time domain signal measured by the dynamic physiological detection instrument is selected from a group consisting of an electrocardiography signal, electroencephalography signal, electromyography signal, plantar pressure signal, and motion characteristic signal measured by an electrocardiogram machine, electroencephalogram machine, electromyogram machine, plantar pressure sensor, inertial measurement system, and a camera device, and a combination thereof.

5. The method for disease risk assessment of claim 3, wherein the characteristic analysis process of the dynamic physiological information is further divided into a plurality of synchronization blocks according to a type or time sequence of events generated when the subject executes the action command, so as to generate a plurality of characteristic indexes based on a correlation, time difference, cycle variation, coordination, and trend of a shifting of a center of gravity between each synchronization block.

6. The method for disease risk assessment of claim 1, wherein in the determining step, when the current data is inputted into the prediction model, the prediction model carries out a weight distribution of the current data and calculation according to a characteristic weight, so as to generate the disease risk assessment result.

7. The method for disease risk assessment of claim 1, wherein the action commands given to the subject is selected from a group consisting of standing on one leg with eyes open, standing on two legs with eyes open, standing on two legs in a tandem stance with eyes open, standing on tiptoes with eyes open, standing on one leg with eyes closed, standing on two legs with eyes closed, standing on two legs in a tandem stance with eyes closed, standing on tiptoes with eyes closed, standing up, sitting down, squatting, walking in a straight line, turning, climbing steps, and a combination thereof.

8. The method for disease risk assessment of claim 7, wherein the dynamic physiological detection instrument is a plantar pressure detector disposed on a shoe sole of the subject, and the action command given to the subject is walking in a straight line, turning, and climbing steps.

9. The method for disease risk assessment of claim 1, wherein in the data acquiring step, the data acquisition module is connected with a healthcare system through the internet for obtaining a date of diagnosis, a name of the diagnosed disease, an International Classification of Diseases Code for the name of the diagnosed disease, a historical Anatomical Therapeutic Chemical Classification code, and a prescription prescribed by a doctor.

10. The method for disease risk assessment of claim 9, wherein after the preprocessing step, the method further comprises a recording step; in the recording step, the current data is stored in a database according to dates as a detection history record of the subject, and the prediction model is established by collecting a pathological data and the detection history record of a plurality of subjects, and using a machine learning algorithm for training and verification; also, the prediction model continues to obtain the pathological data of a plurality of subjects that are diagnosed with diseases diagnosed by medical institutions through a healthcare system in the future, and to obtain the detection history records of the corresponding subjects in the past from the database, whereby the prediction model is updated.
